(19) United States
(12) Patent Application Publication
(10) Pub. No.: US 2012/0202167 A1
(43) Pub. Date: Aug. 9, 2012

(54) DEVICE AND METHOD FOR LASERING BIOLOGICAL TISSUE

(75) Inventor: Anton KASENBACHER, Traunstein (DE)

(73) Assignees: LUMERA LASER GMBH, Kaiserslautern (DE); Dr. Anton Kasenbacher, Traunstein (DE)

(21) Appl. No.: 13/453,485

(22) Filed: Apr. 23, 2012

Related U.S. Application Data
(63) Continuation of application No. 13/064,313, filed on Mar. 17, 2011.
Application No. 13/064,313 is a continuation of application No. PCT/EP2009/006021, filed on Aug. 19, 2009.

(30) Foreign Application Priority Data
Sep. 17, 2008 (DE) 10 2008 047 640.4

(51) Int. Cl. A61C 17/00; A61B 18/20

(52) U.S. Cl. 433/29; 433/217.1; 433/216

(57) ABSTRACT

Device and method for lasering biological tissue. In a general aspect, the device for lasering a biological tissue may include a source configured to provide a pulsed laser beam, an outcoupler configured to couple the laser beam towards the tissue, and an outfeeder configured to feed a photosensitizer in a direction of the tissue where the outfeeder is connected to the outcoupler. In another general aspect, a method for lasering a biological tissue may include applying a photosensitizer towards the tissue, providing a pulsed laser beam, and lasering a site of the tissue with the pulsed laser beam where the laser beam is emitted with a temporal width at a half maximum range from about 1 ps to about 100 ps.

### CROSS-REFERENCE TO RELATED APPLICATIONS

This application is a U.S. continuation application filed under 37 C.F.R. 1.53(b) claiming priority benefit of U.S. application Ser. No. 13/064,313, filed Mar. 17, 2011, pending. This application is a continuation application and claims the benefit under 35 U.S.C. Section 111(a), of PCT International Application No. PCT/EP2009/006021, filed Aug. 19, 2009, which claimed priority to German Application No. DE 10 2008 047 640.4, filed Sep. 17, 2008, the disclosures of which are incorporated herein in its entirety.

### BACKGROUND

1. Field

The invention relates to a device and method for lasering biological tissue, and more particularly, to devices and methods for instant diagnosis and lasering biological tissue.

2. Description of the Related Art

One example of the field of application of the invention is dentistry. In dentistry, a method and a corresponding laser device can be used instead of a mechanical drill for the ablation or abrasion of dentin, particularly dentin infected with caries. However, it is understood that the invention can be applied in lasering other kinds of biological tissue such as hard tissue, soft tissue, and tissue fluids.

In dentistry, particularly in caries therapy, it has been attempted to completely or partially replace the conventional radiographic diagnostic approach and mechanical drill apparatus with a near monochrome (LED) and/or exact monochrome sources of radiation (laser). Potential mutagenic and carcinogenic radiographic radiation is well known in medicine, and this has been the reason why treatments were needed be done under the “as low as reasonably achievable” (ALARA) principle. An ideal alternative would be an analysis without radiographic radiation during treatment if possible. In a practice treating oral tissue structures, the conventional “drill” still remains the main choice in dentistry because of its universality and low investment costs although it potential causes considerable thermo-mechanical damage (frictional heat, cracks, shock waves) coupled with the resulting unavoidable pain. However, there is still no “smart” device for simultaneous and objective detection of pathological structures (e.g., caries) and therapy (e.g., cavitation preparation) with AUTO self-limiting stop for maximum bio-safety. All of these undesirable effects can be avoided by making use of a combined diagnostic and laser device.

Recently, a series of laser systems for dentistry has been tested. However, in many cases, undesirable thermal or other collateral effects were observed, or the ablation efficiency was inadequate. This applied especially to laser systems operating on the basis of pulsed laser beam sources with pulse widths ranging from nano to microseconds. For example, such lasers can be excimer lasers with wavelengths in the ultraviolet range or Er:YSGG (λ=2.7 μm) or Er:YAG lasers (λ=2.94 μm) in the infrared wavelength range. In addition, none of these systems is capable of performing bio-safe detection and therapy.

A substantial advancement was achieved after the introduction of short-pulse laser systems in the picosecond (ps) or femtosecond (fs) range and wavelengths in the visible or near infrared spectral range. First experimental studies indicated that these systems make it possible to achieve high quality dental ablation results with the efficiency at least equal to the performance of a mechanical turbine.

U.S. Pat. No. 5,720,894 describes a method and a device for material ablation by means of a pulsed laser beam source. The ablation parameters to be selected for wavelength, pulse width, energy and repetition rate of the laser pulses are indicated mainly just in reference to the task concerned. Here, each laser pulse is intended to interact with a thin surface portion of the material such that plasma is formed in the focal position of the laser beam. The cited parameters of the laser beam are indicated with a relatively wide range amounting up to 50 mJ or relative to the surface area, up to 15 J/cm². However, particularly when more than three photons are involved, the risk was that such a high pulse energy involving very short laser pulses where the values attained as to power or intensity in the maximum pulse, harmful collateral effects may be materialized due to non-linear processes such as multi-photon ionization. The risk is especially notable when the powerful peak pulses (of a few TW/cm²) that water molecule ionization occurs (ionization energy Eᵢₒₙ=6.5 eV) with fatal collateral effects (i.e. DNA damage and the formation of cavitation bubbles with subsequent unavoidable sonoluminescent fusion in a spectral bandwidth with a range from the ultraviolet (UV) to the radiographic range).

It has been realized that what is needed in order to solve such limitations is to provide a device and a method for lasering biological tissue, which may assure efficient tissue lasering while avoiding or minimizing the damaging effects of tissues being lasered and of the immediate ambience. Also, additional flexibility in selecting the lasering wavelength may be achieved.

In one general aspect, a method for lasering biological tissue may include applying a photosensitizer towards the tissue; providing a pulsed laser beam; and lasering a site of the tissue with the pulsed laser beam, wherein the laser beam being emitted with a temporal width at a half maximum range from about 1 ps to about 100 ps.

In one embodiment of the method for the invention, the method may include a laser pulse repetition rate set between 1 Hz to 1000 kHz. In this arrangement, it may also be provided for that the laser pulses are generated as bursts, each with a predefined number of laser pulses. For example, each site may be lasered with a predefined number of bursts (for example one burst) where the laser pulses may also comprise a pulse peak intensity varying as defined. To advantage no undesirable leading or trailing pulses or underground and offset intensities whatsoever occur before, during or after the burst.

In another embodiment, the energy of the laser pulses may be set with a density ranging from 1.5 J/cm² to 7.5 J/cm², especially in a range below 100 μJ. The focal position of the laser beam on a tissue site may be set on a surface of the tissue with a focusing diameter ranging from 10 to 100 μm.

In another embodiment, the laser pulse peak intensity in lasering a site may range from 10¹¹ to 1.5×10¹² W/cm². In another embodiment, the diagnostic pulse peak intensity when using a pulsed laser beam may range from 10⁶ to 10⁹ W/cm².

In another general aspect, a device for lasering a biological tissue may include a source configured to provide a pulsed laser beam; an outcoupler configured to couple the laser beam towards the tissue; and an outfeeder configured to feed a photosensitizer in a direction of the tissue, wherein the outfeeder being connected to the outcoupler.

The embodiments can be implemented to realize that that when lasering the biological tissue with a laser beam, it is no longer necessary for the tissue itself to be beamed. Instead, the laser beam can be absorbed by substance acting by the absorption as a source of free or quasi-free electrons, and these may communicate the absorbed energy to the material to be ablated. As such, a substance, so called photosensitizer, may be most effectively employed. A photosensitizer may be a chemical light-sensitive compound which may enter into a photochemical reaction after absorption of a light. Activating a photosensitizer can be done by laser light in a suitable wavelength and at adequate intensity. The light absorption may first activate the photosensitizer into a relatively short-lived singlet state which then may be converted into a more stable triplet state. This activated state can then react directly with the material to be ablated.

The embodiments may also be implemented to realize that that laser pulses having a temporal full width at half maximum in the picosecond range can now be used for advantageous effects. This range may provide the ablation efficiency, and the biomedical compatibility can also be optimized due to the optical depth of penetration. Accordingly, the thermal and mechanical stress may be limited.

The embodiments may also be implemented to realize that a marker that may render sites to be lasered or ablated visible for diagnosis can now be implemented simultaneously to the lasering. The marker may be a photosensitizer and/or can be activated by a laser beam or an LED continuously or pulsed with a suitable wavelength, duration and intensity.

The embodiments may also be implemented to realize that a site of the tissue to be lasered or ablated may be encapsulated by integrating an aspiration system in a laser beam decoupler/outcoupler.

The embodiments may be employed for abrading or ablating dentin, particularly when carious. Here, the application may utilize that carious dentin has a porous structure due to bacterial activity. The photosensitizer may gain access through this porous structure in embedding in the carious dentin to be ablated rather than applying to the surface of tissue material to be ablated.

When lasering biological tissue with a short-pulse laser such as a picosecond (ps) or femtosecond (fs) laser, microplasma may be generated within a thin surface layer at the focal position of the laser beam. Here, the microplasma may be ablated in a matter of nanoseconds or microseconds thus the biological tissue may not be ionized by interaction of the laser photons with the quasi-free electrons but minimally invasive thermo-mechanically fragmented. One general intention is to always generate the microplasma in the threshold region, i.e. always below the critical electron density (for the laser wavelength of 1064 nm: 1.03×10²¹ electrons/cm³) so that ablation with maximized medical and biological compatibility may be performed to avoid undesirable collateral effects. Especially, plasma temperatures greater than or equal to 5800 K (surface temperature of the sun) resulting in UV radiation and multiphoton ionization are to be avoided so that water molecules in the tissue are not ionized. In accordance with the present disclosure, an indirect energy input by photosensitizers injection and the usage of picosecond laser pulses provide more biological-medical compatibility. Especially, regarding the stress relaxation, an optical depth of penetration may result in no shock waves and enable treatments to be implemented painlessly.

In general, a surface site of the biological tissue to be treated may be scanned by the laser beam. Where this is concerned, the laser beam may have a top hat profile so that each sub-site focused by the laser beam is scanned with precisely one laser pulse. However, whether a top hat profile is provided or not, it is just as possible to achieve this by defining scanning each adjoining sub-sites with a single laser pulse with an overlap having a surface area smaller than half or smaller than some other fraction of the surface area of a sub-site. This may make it possible when the “cross-section of the laser beam” has a Gauβian profile that a sub-site substantially focused by the laser beam is pulsed substantially by a single laser pulse.

In another embodiment, before applying the photosensitizer, the site to be lasered can be defined by applying a marker to the tissue. Here, the maker may indicate a characteristic stain when in contact with a specific kind of tissue, especially damaged tissue. In this arrangement the marker may involve a photosensitizer thus becoming a diagnostic photosensitizer while the photosensitizer used for ablation can be termed an ablation photosensitizer. However, the marker can also be formed by any other commercially available marker having no photosensitizer response. For example, the ablation photosensitizer has no marker response which means there is no staining effect when coming into contact with the various kinds of tissue.

In still another embodiment, the site to be lasered may be established without the use of a marker by namely detecting the presence or the strength of a signal generated from the tissue. In this arrangement, the signal may be the second or higher harmonic of an electromagnetic radiation directed at the lasering site. Here, the electromagnetic radiation may be a pulsed diagnostic laser beam, the laser pulses of which feature an energy density which is smaller than that needed for lasering the tissue. Indeed, the laser beam and the diagnostic laser beam may be generated by the same laser beam source switched back and forth between two operating modes. More particularly, to distinguish undamaged dentin from carious dentin, the tissue can be activated by the diagnostic laser beam using laser-induced breakdown spectroscopy (LIBS) in the infrared range. Here, a back scattered signal of a second harmonic may indicate healthy tissue (e.g. fibers of collagen capable of mineralization) and the lack of such signal may indicate carious dentin (i.e. irreversibly damaged collagen structures incapable of mineralization). A tissue site can be scanned with the diagnostic laser beam and the data of the backscattered second harmonic can be detected and saved. Based on this data, portions of the site that may require lasering or ablation by the laser beam may be determined.

### BRIEF DESCRIPTION OF THE DRAWINGS

The invention will now be detailed by way of exemplary embodiments with reference to the drawings in which:

FIG. 1 is a diagrammatic illustration of one embodiment of a laser device;

FIG. 2 is a diagrammatic illustration of another embodiment of a laser device;

FIG. 3 is a diagrammatic illustration of one embodiment of a laser beam outcoupler;

FIG. 4 is a diagrammatic illustration of another embodiment of a laser beam outcoupler;

FIG. 5 is a diagrammatic illustration of another embodiment of a laser beam outcoupler;

FIG. 6 is a diagrammatic illustration of another embodiment of a laser beam outcoupler;

FIG. 7 is a diagrammatic illustration of another embodiment of a laser device;

FIG. 8 is a flow chart of one example of an automated combination diagnostic and lasering method; and

FIG. 9 is a flow chart of another example of an automated combination diagnostic and lasering method.

### DESCRIPTION OF EMBODIMENTS

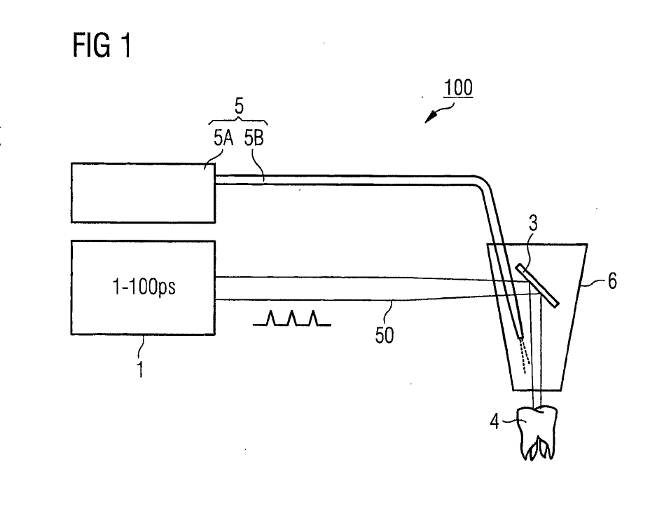

FIG. 1 illustrates one embodiment of a laser device for lasering biological tissue, but not to true scale. FIG. 1 shows a dental laser device for lasering, abrading or ablating dentin, particularly carious dentin. However, the laser device may be any other kind of medical laser device for lasering some other kind of biological tissue.

The laser device 100 comprises a laser beam source 1 that may emit a pulsed laser beam 50 with a laser pulse ranging from 1 to 100 ps. The laser beam may be focused on a patient's tooth 4. It may be necessary to first deflect the laser beam with an optical diverter 3 such as a mirror or deviation prism.

The laser beam source 1 may generate the laser pulses so that the energy per pulse does not exceed 100 pJ. In this case, the focuser 2 for maintaining the energy density values is set so that the laser beam is focused on the surface of the tooth 4 with a diameter range from 10 to 100 μm. The laser beam source 1 may emit the laser pulses with a repetition rate range from 1 Hz to 1000 kHz.

The laser device 100 further comprises an outfeeder 5 for outputting a photosensitizer in the direction of the tooth 4. As shown in FIG. 1, the outfeeder 5 may include a reservoir 5A to house the photosensitizer where the reservoir 5A may be connected to a feeder 5B. The photosensitizer may be erythrosine which can be efficiently activated by two-photon absorption of the laser beam of a Nd:YAG laser (1064 nm) or by one-photon absorption of the frequency doubling component of the Nd:YAG laser (532 nm). For example, photosensitizers may be methylene blue, photofrine, or metalorganic dendrimeres. It is understood that all other photosensitizers referenced in technical literatures even if those are still yet to be developed, may be used if the photosensitizer requires the laser wavelength to be adapted to the corresponding maximum absorption of the photosensitizer or at least in the ambience thereof. The photosensitizers may also be biochemical chromophors. The term photosensitizer may also cover such substances which are not photosensitizers by definition but which may feature properties typical for photosensitizers under defined physical-chemical conditions. Some examples may be any gases, gas mixtures (air), or aerosols if those substances feature photosensitizer properties under defined physical-chemical conditions.

The laser device 100 may also comprise an outcoupler 6 for outcoupling the laser beam 50 in the direction of the tooth 4. As shown in FIG. 1 as an example, the outcoupler 6 may contain a deflector 3 and may be connected with the feeder 5B of the outfeeder 5 so that the photosensitizer, when being applied, may be jetted towards the tooth 4 from the distal end of the outcoupler 6 from the feeder 5B.

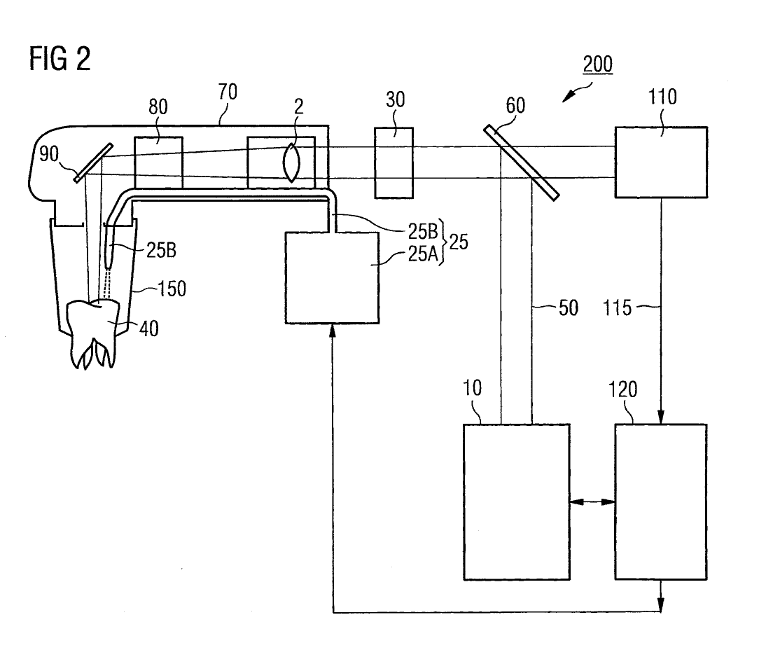

FIG. 2 illustrates another embodiment of a laser device, but not to true scale. The embodiment of a laser device 200 shown in FIG. 2 comprises a laser beam source 10 that may emit a pulsed laser beam 50. In this exemplary embodiment, the laser beam source 10 may be a Nd:YAG laser coupled to a transient or regenerative amplifier emitting laser pulses in a wavelength of 1064 nm. Any other laser beam source such as a Nd:YVO4 or Nd:GdVO4 laser may be used. The pulse duration of the laser pulses may be 10 ps, the repetition rate may range from 1 kHz to 1000 kHz, the energy of the laser pulses may amount to 40 μJ, and whilst at a repetition rate of 100 kHz the mean beam power may be 4 W.

Any other laser may be used as the laser beam source. For example, a diode laser or a diode laser array may be used.

In the embodiment shown in FIG. 2, the laser beam 50 emitted from the laser beam source 10 may be directed at an optical diverter 60 which may selectively divert the laser beam 50 at about 90° at the required wavelength of the laser beam 50. Diverted laser beam 50 may pass through a beam shaper 30 to generate a top hat beam profile.

Then, the laser beam 50 may enter an outcoupler 70 configured as a handpiece fronted by a lens as part of an autofocuser 20, which may ensure that the focal position created by the autofocuser 20 always remains within the plane of the surface of the tooth 40 to be lasered. The autofocuser 20 may be combined with an optical sensing means that senses backscattered radiation from the surface of the tooth 40 to sense whether the surface is still in the focal position of the laser beam. If it is not, a control signal is communicated to the autofocuser 20 for the laser beam to suitably result on the surface of the tooth 40 and return into the focal position of the laser beam by moving the autofocuser 20 forwards or backwards along the propagation path of the laser beam 50. The autofocuser 20 may be moved by a fast stepper motor connected to a carriage mounting the autofocuser 20. However, it is just as possible to configure the autofocuser 20 for its refraction to be tweaked.

FIG. 2 also illustrates how the autofocuser 20 may be arranged so that it focuses the beam on the surface of the tooth 40 with a focal diameter of 40 μm. The laser pulse energy as recited above may result in an energy density of 3.18 J/cm² which may produce a pulse peak intensity of 3.18·10¹¹ W/cm² corresponding to a photon flux density of 1.7·10³⁰photons·cm²·s⁻¹. The electric field strength of the alternating electromagnetic field may be 1.55·10⁷ V/cm and the median electron oscillation energy in the alternating electromagnetic field may amount to 0.021 eV.

It is understood that the beam shaper 30 may also be located in the beam path downstream of the autofocuser 20, particularly in the outcoupler 70 although it is just as possible to combine the autofocuser 20 and beam shaper 30, especially the autofocuser 20 and beam shaper 30 into a common optical component.

The outcoupler 70 may also include a scanner 80 that may scan over a defined site of the surface of the tooth 40 with the laser beam 50 or a diagnostic laser beam by two rotating mirrors, each facing the other. Also, a diverter 90 such as a diverting prism or a reflective mirror may be included to divert the laser beam 50 or a diagnostic laser beam in the direction of the tooth 40.

It is understood that although the scanner 80 is arranged in the handpiece in this embodiment, other embodiments may locate the scanner in the beam path upstream of the handpiece, i.e. particularly within an arm hinging the mirror or at the input thereto upstream of the handpiece.

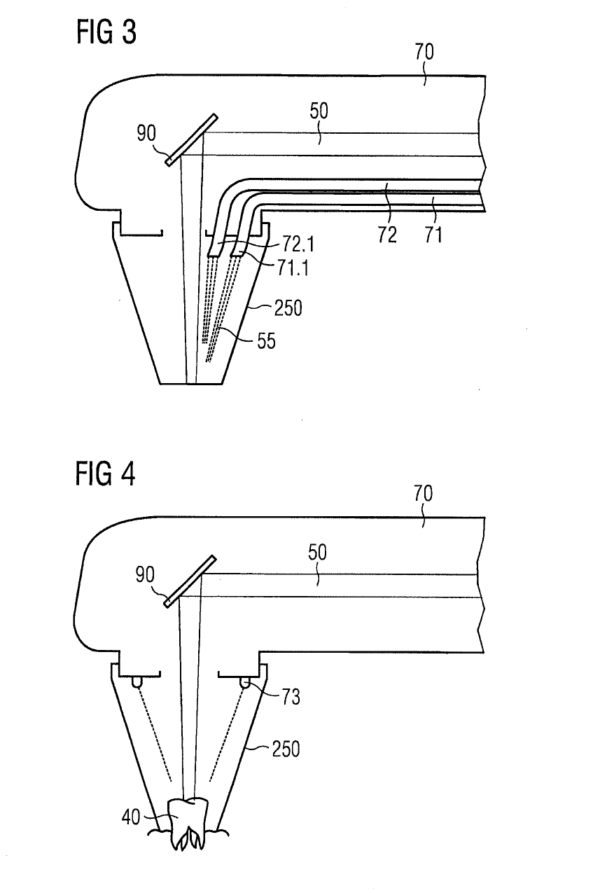

The outcoupler 70 configured as a handpiece may need to be held directed on the tooth being lasered by the physician. In maintaining the position of the distal end of the outcoupler 70 constant relative to the tooth 40, a funnel-shaped locator 150 is secured to the distal end of the outcoupler 70 and can be suitably located on the tooth 40 during lasering as illustrated in FIGS. 3 to 6. A cofferdam or rubber clamp may be placed by the physician to encapsulate the tooth in isolating it from the remaining pharyngeal space.

The laser device 200 may further comprise an outfeeder 25 for outfeeding a photosensitizer in the direction of the tooth 40. The outfeeder 25 may contain a reservoir 25A that is connected to a feeder 25B. The feeder 25B may be ported into the outcoupler 70 and guided within the outcoupler 70 into the locator 150.

The optical or acoustical signals generated from the lasered site of the tooth 40 surface or from the ambience thereof can be detected and used for diagnostic purposes. As explained already, the optical signals may be based, for example, either on the plasma radiation or second harmonic generated (SHG) or higher harmonic generated electromagnetic radiation acting on the dentin involved in lasering. The exemplary aspect as shown in FIG. 2 will now be explained with an example of detecting a SHG signal.

In this mode of diagnosis, a diagnostic laser beam may be emitted like the lasering beam is pulsed for diagnosing whether the sub-site of the dentin is carious or not. Here, the energy or energy density is below the threshold for generating ablation or plasma so that no lasering occurs with the diagnostic laser beam. If not, energy or energy density furnishes a higher SHG signal than carious dentin.

At least some part of the radiation having doubled frequency and being generated from the tooth surface may pass through the laser beam path in the opposite direction, as described above. In other words, the radiation may be diverted by the diverter 90 and pass through the scanner 80 and the autofocuser 20 with the lens to finally incident the optical diverter 60, such as a beam splitter. Here, the beam splitter may be transparent for the wavelength of the SHG signal so that the frequency doubled radiation can be input in an optical detector 110. The optical detector 110 may be a simple photo detector detecting the intensity of the SHG radiation. It is just as possible to use a more complex system such as a spectrometer, CCD camera, or CMOS image sensor as the optical detector 110. Such optical detectors may suitably be used in combination with the autofocuser 20, as already indicated above.

Likewise, the diverter 90 may be engineered to transmit the frequency-doubled radiation generated from the tooth surface and to direct the radiation to the detector 110 with, for example, a glass fiber located downstream of the diverter 90. This may reduce the complexity of the optical beam in transmitting the frequency-doubled radiation since the optics 80, 20, 60, 2 are not designed for several different wavelengths, making them to be coated if necessary. In order to effectively couple the frequency-doubled light, an optical component can be inserted between the diverter 90 and the glass fiber to focus the frequency-doubled light onto the glass fiber. This optical component can be engineered as a microoptical component.

The SHG radiation values detected by the optical detector 110 are converted into a signal 115 and transmitted into a combined analyzer/controller 120, which may also be a computer system for this embodiment. In principle, any other type of control system may be compatible, for instance, memory-programmable controllers, micro controllers, or analog closed-loop controls.

The analyzer/controller 120 can receive a signal containing data as to the operation status of the analyzer/controller 120 from the laser beam source 10. The analyzer/controller may output a control signal to the laser beam source 10 in switching the laser beam source 10, for example, from an idle mode to a lasering mode. Here, the analyzer/controller may function upon receiving the signal 115 communicated by the optical detector 110.

The embodiment shown in FIG. 2 may comprise a laser beam source 10 which is nimble in mode switching “OFF” (idle), “diagnosis”, and “therapy” (lasering) treatment. In this embodiment, the laser beam source 10 may emit both the laser beam required during the “therapy” mode and the diagnostic laser beam required during the “diagnosis” mode with a substantially different energy density per pulse applied to the tooth in W/cm². Here, the energy density applied to the surface of the tooth needs to be reliably below the ablation threshold in the “diagnosis” mode while the energy density is above this threshold in the “therapy” mode.

In a diagnostic mode as described above, a certain surface site of the tooth 40 is scanned with the diagnostic laser beam and the backscattered SHG signal is received and analyzed. This may allow the surface site can be mapped to a certain extent in identifying a portion of the surface to be lasered or ablated. As implementing the diagnostic mode, the analyzer/controller 120 may output a signal to the outfeeder 25 and this signal may allow the feeder 25B and end portion of the controllable nozzle to jet the photosensitizer towards the portion of the tooth surface to be ablated.

FIG. 3 illustrates another example embodiment for a diagnosis. The embodiment illustrated in FIG. 3 includes an outcoupler 70 in the form of a handpiece shown in cross-section. With this particular embodiment, healthy dentin may be distinguished from unhealthy one by means of a marker rather than using a SHG signal. The mark may indicate a characteristic stain when it is in contact with the unhealthy dentin. This marker can be applied to the tissue via a feeder 72 that may also be incorporated within the handpiece as shown in FIG. 3. Once the carious portions of a tissue surface are detected preferably by means of optical imaging with subsequent analysis thereof, photosensitizer is applied to these portions via the feeder 71 for subsequent ablation by the laser beam 50. Accordingly, in this example embodiment, there is no diagnostic laser beam, switching of the laser beam source, or SHG detection. The two feeders 71 and 72 can be used to connect the nozzles 71.1 and 72.1 respectively for a controlled orientation in jetting the materials pin-pointed to the surface of the tissue.

It is to be noted that the embodiment as shown in FIG. 3 may depict a laser beam outcoupler as a stand-alone embodiment. This laser beam outcoupler may comprise a handpiece 70, a diverter 90 for deflecting a lasering beam 50 and/or a diagnostic laser beam, and a locator 250 for locating the handpiece 70 on an ambience of the tissue to be lasered. In this arrangement, the handpiece 70 may be configured so that a photosensitizer can be applied via the feeder 71 incorporated in the handpiece 70 and, where necessary, marker can be jetted via additional feeder 72 on a portion of the tissue to be lasered or diagnosed. It is understood that this separate embodiment can also be combined with any of the other embodiments as described in this application and/or sophisticated with any of the features cited in this application, including also leading devices such as a laser device incorporating a laser beam outcoupler as described above.

Referring now to FIG. 4, an outcoupler 70 in the form of a handpiece, shown in cross-section, illustrates another example embodiment. Here, at least one LED 73 is integrated within the handpiece 70. As shown in the embodiment of FIG. 4, several LEDs 73 may also be incorporated within the handpiece 70 that may serve a physician to illuminate the pharyngeal space when the locator 250 is still to be affixed in place. This may allow the physician to optimally position the locator 250 in relation to the tooth 40 being treated. In addition, these LEDs may also serve to activate a marker applied to the surface of the tooth being treated so that the carious locations may indicate a characteristic stain. The image created by the marker in this way can be scanned by the same optics used to incouple the laser beam 50. On the basis of this imaging, the photosensitizer can be applied to the sites to be lasered or ablated. The LEDs 73 may be arranged on a horizontal end portion of the handpiece 70. For example, The LEDs 73 may be arranged in a circle to achieve illumination as best possible homogenous and rotationally symmetrical. The LEDs 72 may be connected by leads (not shown) integrated within the handpiece 70 for powering the LED 73. The LEDs 73 may be LEDs emitting light in a single color, for example, red, such as quasi-monochromatic LEDs. However, white light LEDs could be used for a better illumination of the pharyngeal space and circumstances so that a larger choice of markers for activation at differing wavelengths is available.

It is to be noted that the embodiment as shown in FIG. 4 may depict a laser beam outcoupler as a stand-alone embodiment. This laser beam outcoupler may comprise a handpiece 70, a diverter 90 for deflecting a laser beam 50 and/or a diagnostic laser beam, and a locator 250 for locating the handpiece 70 on an ambience of the tissue to be lasered. This laser beam outcoupler may further comprise at least one LED 73 for illuminating and/or activating a marker or photosensitizer. It is understood that this separate embodiment can also be combined with any of the other embodiments as described in this application and/or sophisticated with any of the features cited in this application, including also leading devices such as a laser device incorporating a laser beam outcoupler as described above.

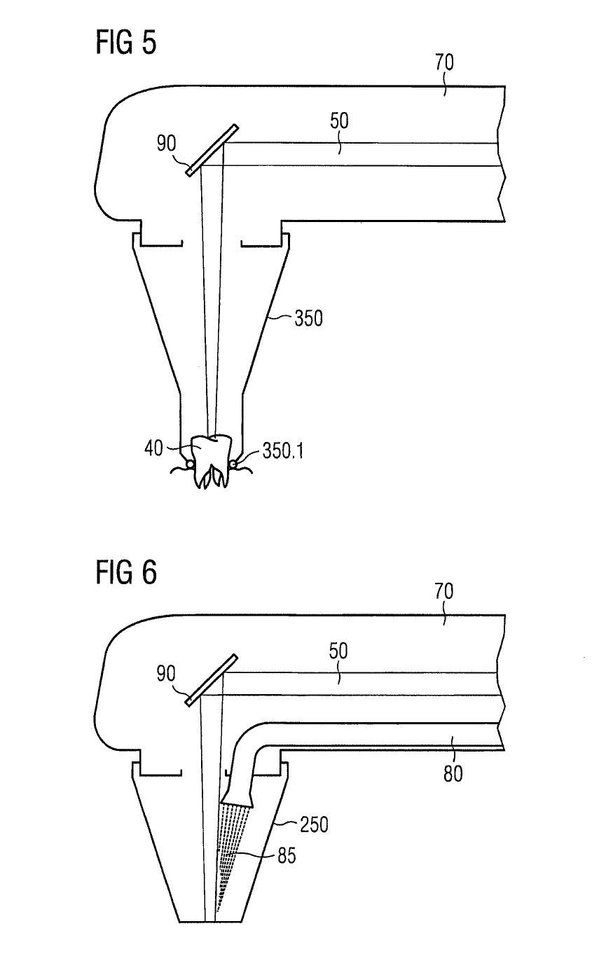

Referring now to FIG. 5, an outcoupler 70 in the form of a handpiece, shown in cross-section, illustrates another example embodiment. Here, the handpiece 70 may feature a locator 350 having an encapsulating function in addition to a locating function of the tooth 40. As illustrated in the exemplary embodiment of FIG. 5, the seal 350.1 may be applied to the bottom rim of the locator 350. Here, the seal 350.1 is indicated simply symbolically and not necessarily to be appreciated as being technically realistic. One object of such a locator may be to encapsulate the direct vicinity of the tooth 40 being treated at best air- and gas-tight from the remaining pharyngeal space. Such an encapsulated location of this kind may allow to optimize the treatment of the tooth in a wide variety of ways as will now be explained with the following example aspects. For example, an aspirator may be integrated within the handpiece 70 to allow the locator to seal off the site from the outside and this may result efficient and reliable removal of the ablated debris. In addition, a controlled atmosphere can be created surrounding the tooth 40.

It is to be noted that the embodiment as shown in FIG. 5 may depict a laser beam outcoupler as a stand-alone embodiment. This laser beam outcoupler may comprise a handpiece 70, a diverter 90 for deflecting a lasering beam 50 and/or a diagnostic laser beam, and a locator 350 for locating the handpiece 70 on an ambience of the tissue to be lasered. In this arrangement, the locator 350 may be designed to seal and encapsulate a tissue site to be lasered. It is understood that this separate embodiment can also be combined with any of the other embodiments as described in this application and/or sophisticated with any of the features cited in this application, including also leading devices such as a laser device incorporating a laser beam outcoupler as described above.

Referring now to FIG. 6, an outcoupler 70 in the form of a handpiece, shown in cross-section, illustrates another exemplary embodiment. Here, the handpiece 70 may mount a locator 250 and may be configured to integrate an aspirator duct 80 for efficient aspiration of the ablated debris in tissue treatment. The aspirator duct 80 may be connected to an aspirator system (not shown) integrated in the handpiece 70. An open end of the aspirator duct protruding into the locator 250 such that it is directed at the site being lasered to aspirate the ablated debris materializing in lasering. The end of the aspirator duct 80 may be mounted movable, for example by user's control and orientation. This also includes varying spacing between the aspirator duct 80 and the site being lasered.

It is to be noted that the embodiment as shown in FIG. 6 may depict a laser beam outcoupler as a stand-alone embodiment. This laser beam outcoupler may comprise a handpiece 70, a diverter 90 for deflecting a lasering beam 50 and/or a diagnostic laser beam, and a locator 250 for locating the handpiece 70 on an ambience of the tissue to be lasered. Here, the handpiece 70 and locator 350 may be configured so that an aspirator duct 80 is integrated therein and the end of the duct can be directed at the tissue site being lasered. It is understood that this separate embodiment can also be combined with any of the other embodiments as described in this application and/or sophisticated with any of the features cited in this application, including also leading devices such as a laser device incorporating a laser beam outcoupler as described above. Especially, a combination of the embodiments as illustrated in FIGS. 5 and 6, i.e. an encapsulated sealed attachment to an aspirator system may allow potentially toxic lasering. For example, the ablation of amalgam fillings can be performed, in which case the gas-tight encapsulation may make it safe to remove the ablated debris, essentially elementary mercury with practically no remainders. As described in this application, laser ablation of the amalgam filling could be performed with the assistance of a photosensitizer. Thus, this embodiment may allow performing amalgam removal by lasering in compliance with the maximum workplace concentration (MAK) as required by law for mercury vapors.

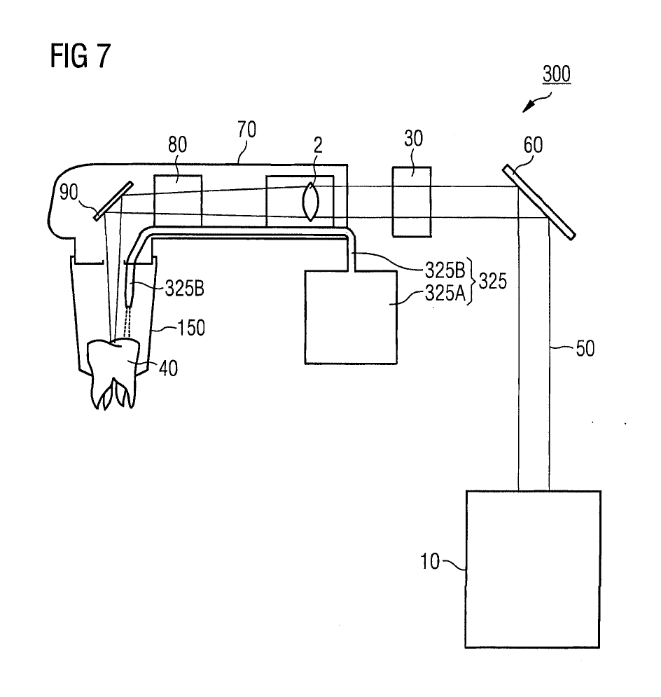

FIG. 7 illustrates a further embodiment of a laser device not shown true to scale. The embodiment of a laser device 300 shown in FIG. 7 comprises substantially the same components as the components of exemplary embodiment described in FIG. 2 which are identified with the same reference numerals. However, unlike the laser device illustrated in FIG. 2, the laser device 300 may feature a generator 325 comprising a reservoir 325A connected to the handpiece 70 by a feeder 325B. The feeder 325B may be integrated through the handpiece to the locator 150 and may feature an orifice directed at the tooth being lasered at the end of the feeder. Here, the generator 325 shown in FIG. 7 does not illustrate its detailed features but the generator 325 may have various functions. For example, the generator 325 may serve predominantly to create a certain atmosphere in the ambience of the tooth 40 being treated.

In one simple variant, vacuum atmosphere can be generated by the generator 325 comprising a vacuum pump. In this example, the locator 150, like the locator 350 of the embodiment described in FIG. 5, may be configured as an encapsulating locator. In addition, the locator 150 may be—when wanted or necessary—sealed off from the handpiece 70 by disposing a window transparent to the lasering beam 50 between the handpiece 70 and the locator 150. In a somewhat less complicated variant, when vacuum atmospheres are needed to be created above the tooth 40, there may be no seal or at least none-complete seal provided between the handpiece 70 and the locator 150. The generator 325 may also be designed to create a positive pressure. Furthermore, the generator 325 may be designed to create a specific gas atmosphere in the ambience of the tooth 40 such as furnishing a gas such as O₂, N₂, H₂O (water vapor) or some rare gas. Especially when ablating amalgam fillings, utilizing the generator can be advantageous in binding the ablated mercury in a certain way to remove amalgam fillings from the ambience of the tooth 40. The generator 325 may also be designed to cool the tooth 40 by generating a cooling medium by jetting cooling air on to the ablated surface site. The generator 325 may also be designed as an aerosol generator that may generate a gas in which particles such as microscopic (nano) or macroscopic particles are dispersed in handling certain functions for the ablation. These particles may have a cooling function. In addition to this, the analyzer/controller 120 and the detector 110 of the embodiment described in FIG. 2 may be included in this particular embodiment described in FIG. 7. Here, the analyzer/controller 120 may also be connected to the generator 325 so that the analyzer/controller 120 may control the generator 325.

It is to be noted that the embodiment as shown in FIG. 7 may depict a laser device as a stand-alone embodiment. This laser device may comprise a source 10 for furnishing a lasering beam 50, an outcoupler 70 for outcoupling the lasering beam 50 in the direction of the tissue site being lasered, and a generator 325 for generating or furnishing an atmosphere in an ambience of the tissue being lasered. It is understood that this stand-alone embodiment can also be combined with any of the other embodiments as described in this application and/or sophisticated with any of the features cited in this application.

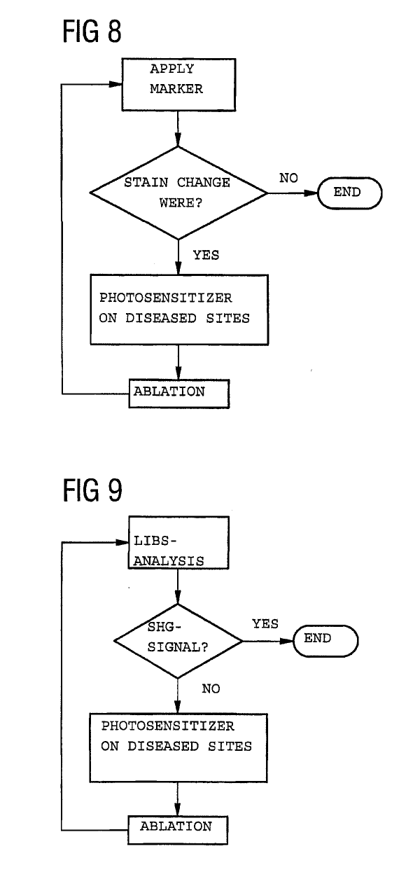

FIG. 8 illustrates a flow chart for one example of methods of an automated combination ablation and diagnostic process when using a marker. In step S1, a marker may be applied (S1). Then, it is established whether a change in stain has been occurred, indicating damaged tissue (S2). If no change in stain is detected, the process may be discontinued. The changes in stain may be detected with a spatial resolution of the surface being imaged on a detector such as a CCD or CMOS element. Here, the changes may be detected by scanning the image and electronically storing the result of the spatial resolution. Then, the marker may be removed and a photosensitizer may be applied to the sites detected as damaged (S4). The, the ablation may be done by the laser beam (S5). Here, the parameters such as, but not limited to, duration or power of the lasering may be previously set by the user. After this, the process may repeat from S1.

Now, FIG. 9 illustrates a flow chart for one example of methods of an automated combination ablation and diagnostic process using LIBS technology. In step s1, a site may be scanned with a diagnostic laser beam and simultaneously the detection of a SHG signal may be performed as described for the embodiment illustrated in FIG. 2 (S1). Then, it is established which sites may be viewed as healthy by detecting a backscattered SHG signal from the site. When an SHG signal is returned from all of the surface, the process may be discontinued. Thus, establishing which sites are healthy may be performed with a spatial resolution. Here, the complementary sites can be electronically stored as being diseased and a photosensitizer may be applied to such sites (S4). Then, the ablation is performed with the laser beam (S5). Here, the parameters such as, but not limited to, duration or power of the lasering may be previously set by the user. After this, an LIBS analysis may be repeated from S1.

It is to be noted that the embodiments as shown in FIGS. 8 and 9 may depict a combined lasering and diagnosis process as a stand-alone embodiment. The embodiments may comprise: detecting diseased sites by means of marker or LIBS, applying a photosensitizer to the diseased sites, ablating the diseased sites by means of a laser beam, and repeating detection of any remaining disease and application of photosensitizer until no more disease is detected. It is understood that each of these stand-alone embodiments can also be combined with any of the other embodiments as described in this application and/or sophisticated with any of the features cited in this application.

It is again to be understood that all features described in the detailed embodiments and stand-alone embodiments may also be applicable to any other embodiments and stand-alone embodiments as described. Also, it may be pointed out that the above embodiments are exemplary, and that the disclosure of this application also covers the combinations of features which are described in different exemplary embodiments, to the extent that this is technically possible.

What is claimed is:

1. A method for ablating a biological tissue, comprising:
applying a photosensitizer towards the tissue;
providing a pulsed laser beam; and
ablating the tissue with the pulsed laser beam by directing the pulsed laser beam onto a site of the tissue where the photosensitizer is applied, the laser beam being emitted with a temporal width at a half maximum range from about 1 picosecond to about 100 picoseconds.

2. The method of claim 1, wherein
the laser beam has a laser pulse wavelength,
the laser pulse wavelength being set so that at least part of the laser beam is absorbed by a two-photon absorption in the photosensitizer, and
the laser beam being absorbed near to an absorption maximum of the photosensitizer.

3. The method of claim 1, wherein
the laser beam has a laser pulse wavelength,
the laser pulse wavelength being set so that at least part of the laser beam is absorbed by a one-photon absorption in the photosensitizer, and
the laser beam being absorbed near to an absorption maximum of the photosensitizer.

4. The method of claim 1, wherein a laser pulse repetition rate is set with a range from about 1 Hz to about 1000 kHz.

5. The method of claim 1, wherein the method is employed for ablation or abrasion of dentin.

6. The method of claim 1, wherein the laser beam comprises a top hat beam profile.

7. The method of claim 1, wherein the lasering site is scanned by the laser beam.

8. The method of claim 7, wherein the laser beam lasers at least one sub-site, the sub-site being focused by precisely one laser pulse.

9. The method of claim 8, wherein the sub-sites overlap provides an overlapping area, and the overlapping area has a first surface area smaller than one half of a second surface area of the sub-site.

10. The method of claim 1, wherein the lasering a site of the tissue further comprises controlling the laser beam to remain on a surface of the site.

11. The method of claim 1, wherein the site is defined by applying a marker to the tissue, the marker indicating a characteristic stain when in contact with the tissue requiring a treatment.

12. The method of claim 1, wherein the site is established by detecting at least one of a presence and a strength of a signal generated from the tissue.

13. The method of claim 12, wherein the signal is at least one of a second harmonic and a higher harmonic of an electromagnetic radiation directed at the site.

14. The method of claim 13, wherein the electromagnetic radiation being at least one of a particularly pulsed diagnostic laser beam radiation and a laser pulse radiation features a first energy density smaller than a second energy density needed for lasering the tissue.

15. The method of claim 14, wherein the laser beam and the diagnostic laser beam are generated by one and a same laser beam source.

16. A device for ablating a biological tissue, comprising:
a source configured to provide a pulsed laser beam;
an outcoupler configured to couple the laser beam towards the tissue; and
an outfeeder configured to feed a photosensitizer in a direction of the tissue, the outfeeder being connected to the outcoupler, wherein
the source is configured to provide the laser beam having parameters so as to ablate the tissue underneath an area fed by the photosensitizer.

17. The device of claim 16, wherein the device is a dental lasering device for at least ablating and abrasion of dentin.

18. The device of claim 16, wherein the source provides the pulsed laser beam with a pulse range from about 1 picosecond to about 100 picoseconds.

19. The device of claim 16, wherein the source provides the pulsed laser beam with a pulse repetition rate range from about 1 Hz to about 1000 kHz.

20. The device of claim 16, wherein
the laser beam has a laser pulse wavelength,
the laser pulse wavelength being set so that at least part of the laser beam is absorbed by a two-photon absorption in the photosensitizer, and
the laser beam being absorbed near to an absorption maximum of the photosensitizer.

21. The device of claim 16, wherein
the laser beam has a laser pulse wavelength,
the laser pulse wavelength being set so that at least part of the laser beam is absorbed by a one-photon absorption in the photosensitizer, and
the laser beam is absorbed near to an absorption maximum of the photosensitizer.

22. The device of claim 16, further comprising:
a locator connected to the laser beam outcoupler, the locator being configured to locate a distal end of the laser beam outcoupler relative to a portion of the tissue.

23. The device of claim 16, further comprising:
a beam shaper configured to produce a top hat beam profile of the pulsed laser beam, the beam being arranged in a path of the laser beam.

24. The device of claim 16, further comprising a scanner configured to scan a site of the tissue with the laser beam.

25. The device of claim 24, wherein the scanner is engineered for a sub-site focused by the laser beam to be lasered by precisely one laser pulse.

26. The device of claim 24, wherein the scanner is engineered for each adjoining sub-site to be lasered with a single laser pulse with an overlap having a surface area smaller than half a sub-site.

27. The device of claims 16, further comprising an autofocuser to maintain a focal position of the laser beam on a surface of the tissue.

28. The device of claim 16, further comprising a detector configured to detect at least one of a presence and a strength of a signal, the signal being generated in at least one of the tissue and an ambience.

29. The device of claim 28, wherein the detector comprises an optical sensor.

30. The device of claim 29, wherein the optical sensor is designed to sense at least one of a second harmonic and higher harmonic of an electromagnetic radiation beamed into the tissue.

31. The device of claim 16, wherein the outcoupler is provided in a form of a handpiece, and a portion of the outfeeder is contained therein.

32. The device of claim 31, further comprising:
a scanner configured to scan a site of the tissue with the laser beam; and
an autofocuser to maintain a focal position of the laser beam on a surface of the tissue, at least one of the scanner and the autofocuser being arranged in the handpiece.
